(19) United States
(12) Patent Application Publication
(10) Pub. No.: US 2024/0115150 A1
(43) Pub. Date: Apr. 11, 2024

(54) BIOMETRIC INFORMATION DETECTION SYSTEM, BIOMETRIC INFORMATION DETECTION METHOD, AND INFORMATION STORAGE MEDIUM

(71) Applicant: Sekisui House, Ltd., Osaka (JP)

(72) Inventors: Akihiro FUKUSHIMA, Osaka (JP); Buntoku MORI, Osaka (JP); Masae NAKAMOTO, Osaka (JP)

(21) Appl. No.: 18/477,559

(22) Filed: Sep. 29, 2023

(30) Foreign Application Priority Data
Oct. 6, 2022 (JP) 2022-161840

(51) Int. Cl. A61B 5/0507; G01S 7/41

(52) U.S. Cl. CPC A61B 5/0507; G01S 7/415

(57) ABSTRACT

To stably obtain biometric information, such as a respiratory rate and a heart rate, from a time waveform indicating a change in phase of a Doppler signal, provided is a biometric information detection system including: an arctangent demodulation module which acquires a time waveform indicating a change in phase of a Doppler signal acquired by receiving a wave reflected from a person to be measured; a unit waveform acquisition module which acquires a plurality of unit waveforms from the time waveform; a reference waveform generation module which generates a reference waveform based on the plurality of unit waveforms; a waveform correction module which corrects the time waveform based on the reference waveform; and a biometric information acquisition module which acquires biometric information based on the corrected time waveform.

### CROSS-REFERENCE TO RELATED APPLICATION

The present application claims priority from Japanese application JP 2022-161840 filed on Oct. 6, 2022, the content 10 of which is hereby incorporated by reference into this application.

### BACKGROUND OF THE INVENTION

### 1. Field of the Invention

The present invention relates to a biometric information detection system, a biometric information detection method, and an information storage medium, and more particularly, to a technology of detecting biometric information, such as a heart rate and a respiratory rate, through use of arctangent demodulation.

### 2. Description of the Related Art

Various systems for detecting biometric information, such as a heart rate and a respiratory rate, of a person to be measured have been considered. In view of a load imposed on the person to be measured, a method of measuring a heart rate in a non-contact manner through use of a microwave Doppler sensor as described in Japanese Patent Application Laid-open No. 2017-134795 is considered as promising. With the microwave Doppler sensor, the biometric information can be acquired by measuring movement of a body surface of the person to be measured or movement within a body of the person to be measured.

A Doppler signal output from the Doppler sensor includes an I component and a Q component, and it is known that a change in phase obtained when those components are mapped on an IQ plane well represents the biometric information, such as the heart rate and the respiratory rate. Thus, as described in Heesoo Kim and Jinho Jeong, “Non-Contact Measurement of Human Respiration and Heartbeat Using W-band Doppler Radar Sensor,” Sensors 2020, Sep. 12, 2020, acquisition of a time waveform derived from respiration or heartbeat from a change in phase of the Doppler signal through use of a method called “arctangent demodulation” is often conducted. The heart rate or the respiratory rate of the person to be measured can be obtained by counting the number of peaks per unit time in such a time waveform.

However, the Doppler signal includes various noise components. Accordingly, it is sometimes difficult to detect the biometric information from the time waveform indicating a change in phase of the Doppler signal. That is, when an amplitude of a portion that is exhibited in the time waveform and is derived from heartbeat or respiration is small, it is difficult to discriminate whether or not the portion is a peak to be counted, and it consequently becomes difficult to accurately detect the biometric information, such as the heart rate or the respiratory rate.

### SUMMARY OF THE INVENTION

The present invention has been made in view of the above-mentioned problem, and has an object to provide a biometric information detection system, a biometric information detection method, and an information storage medium with which biometric information, such as a respiratory rate and a heart rate, can be stably obtained from a time waveform indicating a change in phase of a Doppler signal.

(1) According to at least one embodiment of the present invention, there is provided a biometric information detection system including: a demodulation module configured to acquire a time waveform indicating a change in phase of a Doppler signal; a unit waveform acquisition module configured to acquire a plurality of unit waveforms from the time waveform; a reference waveform generation module configured to generate a reference waveform based on the plurality of unit waveforms; a correction module configured to correct the time waveform based on the reference waveform; and a biometric information acquisition module configured to acquire biometric information based on the corrected time waveform.

(2) In the biometric information detection system according to Item (1), the unit waveform acquisition module may be configured to acquire the plurality of unit waveforms based on a time that gives one of a local maximum value or a local minimum value of the time waveform.

(3) In the biometric information detection system according to Item (1) or (2), the reference waveform generation module may be configured to generate the reference waveform by averaging the plurality of unit waveforms.

(4) In the biometric information detection system according to any one of Items (1) to (3), the correction module may be configured to correct the biometric information by convolution integration which uses the time waveform and the reference waveform.

(5) In the biometric information detection system according to any one of Items (1) to (4), the reference waveform generation module may be configured to exclude some of the plurality of unit waveforms based on a time width of each of the plurality of unit waveforms, and generate the reference waveform based on remaining unit waveforms among the plurality of unit waveforms.

(6) In the biometric information detection system according to Item (5), the reference waveform generation module may be configured to output an alert depending on a ratio of a number of unit waveforms that are excluded to a number of unit waveforms that are acquired by the unit waveform acquisition module.

(7) According to at least one embodiment of the present invention, there is provided a biometric information detection method including: acquiring a time waveform indicating a change in phase of a Doppler signal; acquiring a plurality of unit waveforms from the time waveform; generating a reference waveform based on the plurality of unit waveforms; correcting the time waveform based on the reference waveform; and acquiring biometric information based on the corrected time waveform.

(8) According to at least one embodiment of the present invention, there is provided an information storage medium storing a program for causing a computer to execute: acquiring a time waveform indicating a change in phase of a Doppler signal; acquiring a plurality of unit waveforms from the time waveform; generating a reference waveform based on the plurality of unit waveforms; correcting the time waveform based on the reference waveform; and acquiring biometric information based on the corrected time waveform.

According to the at least one embodiment of the present invention, the biometric information, such as a respiratory rate and a heart rate, can be stably obtained from the time waveform indicating the change in phase of the Doppler signal.

### BRIEF DESCRIPTION OF THE DRAWINGS

FIG. 1 is a configuration diagram of a biometric information detection system according to at least one embodiment of the present invention.

FIG. 2 is a functional block diagram of a signal processing device.

FIG. 3 is a graph for showing respiratory data obtained by arctangent demodulation.

FIG. 4A is a graph for showing a respiratory waveform before the convolution processing.

FIG. 4B is a graph for showing a respiratory waveform after the convolution processing.

### DETAILED DESCRIPTION OF THE INVENTION

Now, at least one embodiment of the present invention is described in detail with reference to the accompanying drawings.

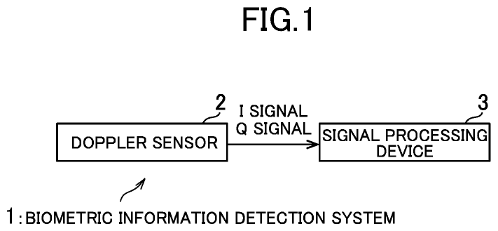

FIG. 1 is a configuration diagram of a biometric information detection system according to the at least one embodiment of the present invention. As illustrated in FIG. 1, the biometric information detection system 1 includes a Doppler sensor 2 and a signal processing device 3. The biometric information detection system 1 is set in a house, for example, and detects, for example, a respiratory rate per unit time of a person to be measured who is sleeping. The Doppler sensor 2 is installed toward a chest of the person to be measured in the vicinity of a bed, for example. A microwave is emitted from the Doppler sensor 2, and the wave reflected by the chest of the person to be measured is received by the Doppler sensor 2. With the Doppler effect, the reflected wave is shifted in frequency, and a heart rate of the person to be measured can be obtained by observing the reflected wave. The reflected wave is detected as a Doppler signal including an I signal being an in-phase component with respect to the transmitted wave and a Q signal being a quadrature component with respect to the transmitted wave, and is output to the signal processing device 3 in a digital form. The Doppler signal input to the signal processing device 3 is time-series data, and indicates an amplitude at each time (I component and Q component).

It is only required that the signal processing device 3 be formed of a publicly-known computer including, for example, a CPU, a memory, an input device, and a display, and the signal processing device 3 generates the respiratory rate of the person to be measured based on the Doppler signal output from the Doppler sensor 2.

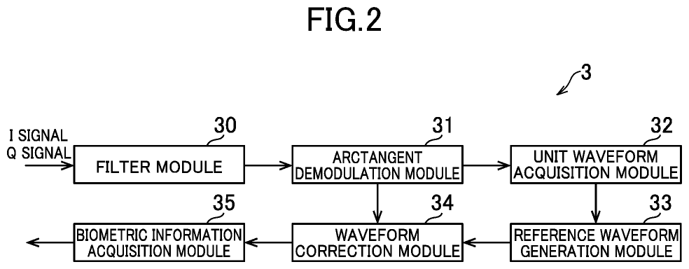

FIG. 2 is a functional block diagram of the signal processing device 3. As illustrated in FIG. 2, the signal processing device 3 includes a filter module 30, an arctangent demodulation module 31, a unit waveform acquisition module 32, a reference waveform generation module 33, a waveform correction module 34, and a biometric information acquisition module 35. Those functional blocks are implemented when a signal processing program is executed by the signal processing device 3, which is the computer. The signal processing program may be stored in one of various computer-readable information storage media such as a semiconductor memory, and may be read out from the medium onto the signal processing device 3. As another example, the signal processing program may be downloaded onto the signal processing device 3 via a data communication line such as the Internet.

The filter module 30 applies filter processing for removing noise to each of pieces of data on the I component and the Q component of the Doppler signal. The filter module 30 may be a low-pass filter, for example.

The arctangent demodulation module 31 applies a predetermined demodulation algorithm to the Doppler signal from which the noise has been removed, to thereby acquire a time waveform (respiratory waveform) indicating a change in phase of the Doppler signal. For example, publicly-known arctangent demodulation is adopted to obtain an arctangent of a value obtained by dividing the Q component of the Doppler signal from which the noise has been removed by the I component thereof, to thereby be able to suppress an amplitude component of the Doppler signal to acquire the time waveform indicating a change in phase of the Doppler signal. The acquired time waveform may be corrected in various manners. Various publicly-known improved algorithms exist for the arctangent demodulation, but the time waveform may be acquired through use of any algorithm. For example, the algorithm as described in Heesoo Kim and Jinho Jeong, “Non-Contact Measurement of Human Respiration and Heartbeat Using W-band Doppler Radar Sensor,” Sensors 2020, Sep. 12, 2020 may be adopted.

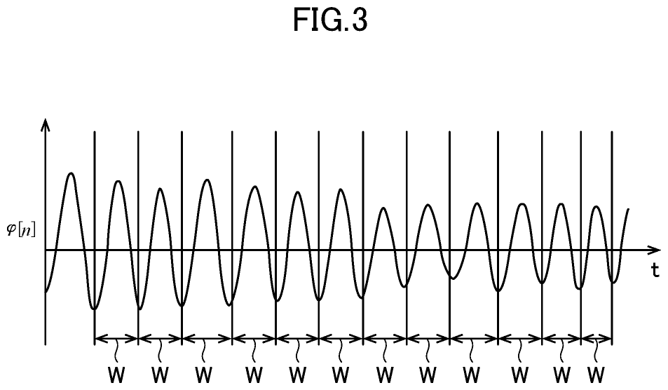

FIG. 3 shows an example of the time waveform obtained by the arctangent demodulation module 31. An interval from a local minimum value to the next local minimum value in the time waveform shown in FIG. 3 indicates one breath of the person to be measured. Accordingly, the respiratory rate of the person to be measured can be obtained by counting the number of peaks per unit time (one minute) in the time waveform shown in FIG. 3.

The unit waveform acquisition module 32 acquires a plurality of unit waveforms from the time waveform output from the arctangent demodulation module 31. In this case, the unit waveform acquisition module 32 cuts out, from the time waveform indicating the respiration, a portion of an interval from a time that gives the local minimum value thereof to a time that gives the next local minimum value, and acquires the cut-out portions as the unit waveforms. That is, the unit waveform acquisition module 32 cuts out a waveform portion corresponding to each interval W of FIG. 3, and acquires the cut-out waveform portions as the unit waveforms. It should be noted, however, that respiratory motions of the person to be measured slightly differ from one to another in general, and hence each of the unit waveforms is not necessarily the same as another unit waveform. While the interval W from the local minimum value to the next local minimum value is cut out in this case, a waveform portion corresponding to the interval W from the local maximum value to the next local maximum value may be cut out.

The reference waveform generation module 33 generates a reference waveform based on the unit waveforms acquired by the unit waveform acquisition module 32. For example, the reference waveform may be generated by averaging the unit waveforms. Specifically, the reference waveform may be obtained by matching starting points or peaks of the respective unit waveforms with each other and adding and averaging values of all the unit waveforms. The reference waveform may be generated by any other method as long as a waveform having a shape corresponding to a statistical representative of the unit waveforms acquired by the unit waveform acquisition module 32 is obtained. The reference waveform generation module 33 is not required to use all of the unit waveforms acquired by the unit waveform acquisition module 32. For example, some of the unit waveforms may be excluded based on a time width of each unit waveform, and the reference waveform may be generated from the remaining unit waveforms. Assuming that a lower limit of the number of breaths per minute of humans is 3 as an example and an upper limit thereof is 40 as an example, a unit waveform having a time width of 20 seconds or longer, or a unit waveform having a time width of 1.5 seconds or shorter may be excluded to generate the reference waveform. As another example, when a time width of a unit waveform among the unit waveforms corresponds to a statistical outlier, such a unit waveform may be excluded. Further, the reference waveform generation module 33 may calculate a ratio of the number of unit waveforms that have been excluded as described above to the number of unit waveforms that have been acquired by the unit waveform acquisition module 32, and when a value of the ratio is equal to or larger than a predetermined threshold value, the reference waveform generation module 33 may output an alert indicating the fact. With this configuration, it is possible to notify a user that there is a possibility that a likely respiratory rate cannot be output.

The waveform correction module 34 corrects the time waveform output from the arctangent demodulation module 31 based on the reference waveform. In particular, the time waveform is corrected in such a manner that a waveform portion similar to the reference waveform is emphasized. Specifically, the corrected time waveform is acquired by executing convolution integration which involves convolving the time waveform with the reference waveform.

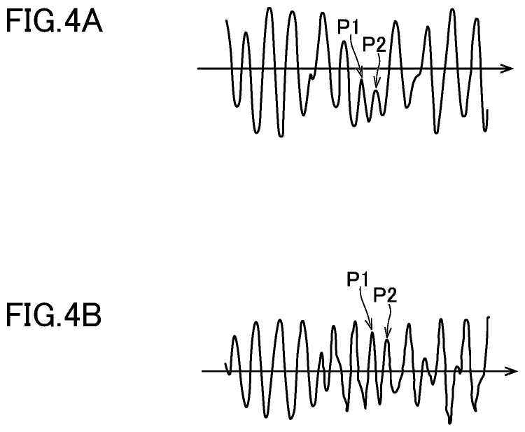

FIG. 4A and FIG. 4B are graphs for showing convolution processing, in which FIG. 4A shows a waveform before the convolution processing and FIG. 4B shows a waveform after the convolution processing. In the waveform before the convolution processing, peaks each having a negative local maximum value exist as indicated by reference symbols P1 and P2, but in the waveform after the convolution processing, the waveform is corrected in such a manner that any of the peaks have a positive local maximum value.

The biometric information acquisition module 35 acquires the respiratory rate of the person to be measured based on data on the waveform corrected by the waveform correction module 34. Specifically, the number of peaks each having the local maximum value that is larger than 0 per unit time (for example, one minute) is counted, and the obtained number of peaks is set as the respiratory rate. According to the at least one embodiment, as shown in FIG. 4B, the height of each of the small peaks as indicated by the reference symbols P1 and P2 is increased by the waveform correction module 34, and hence a waveform portion similar to the reference waveform can be reliably counted.

According to the biometric information detection system 1 described above, even in a case in which the time waveform indicating the respiration of the person to be measured includes a small peak, such a small peak can be emphasized when the peak is similar to the reference waveform. With this configuration, a peak derived from the respiration can be reliably counted, and hence an accurate respiratory rate of the person to be measured can be obtained.

The present invention is not limited the at least one embodiment described above, and various modifications can be made thereto. For example, in the above description, the respiratory rate is detected as the biometric information of the person to be measured, but the heart rate can also be detected in a similar manner.

While there have been described what are at present considered to be certain embodiments of the invention, it will be understood that various modifications may be made thereto, and it is intended that the appended claims cover all such modifications as fall within the true spirit and scope of the invention.

What is claimed is:

1. A biometric information detection system, comprising:
at least one processor; and
at least one memory device storing instructions which, when executed by the at least one processor, causes the at least one processor to perform operations including:
acquiring a time waveform indicating a change in phase of a Doppler signal acquired by receiving a wave reflected from a person to be measured;
acquiring a plurality of unit waveforms from the time waveform;
generating a reference waveform based on the plurality of unit waveforms;
correcting the time waveform based on the reference waveform; and
acquiring biometric information based on the corrected time waveform.

2. The biometric information detection system according to claim 1, wherein the plurality of unit waveforms is acquired based on a time that gives one of a local maximum value or a local minimum value of the time waveform.

3. The biometric information detection system according to claim 2, wherein the biometric information is corrected by convolution integration which uses the time waveform and the reference waveform.

4. The biometric information detection system according to claim 2, wherein the reference waveform is generated by averaging the plurality of unit waveforms.

5. The biometric information detection system according to claim 4, wherein the biometric information is corrected by convolution integration which uses the time waveform and the reference waveform.

6. The biometric information detection system according to claim 1, wherein
the operations further comprise excluding some of the plurality of unit waveforms based on a time width of each of the plurality of unit waveforms; and
the reference waveform is generated based on remaining unit waveforms among the plurality of unit waveforms.

7. The biometric information detection system according to claim 6, wherein the operations further comprise outputting an alert depending on a ratio of a number of unit waveforms that are excluded to a number of acquired unit waveforms.

8. A biometric information detection method, comprising:
acquiring a time waveform indicating a change in phase of a Doppler signal acquired by receiving a wave reflected from a person to be measured;
acquiring a plurality of unit waveforms from the time waveform;
generating a reference waveform based on the plurality of unit waveforms;
correcting the time waveform based on the reference waveform; and
acquiring biometric information based on the corrected time waveform.

9. An information storage medium storing a program for causing a computer to execute:
acquiring a time waveform indicating a change in phase of a Doppler signal acquired by receiving a wave reflected from a person to be measured;
acquiring a plurality of unit waveforms from the time waveform;
generating a reference waveform based on the plurality of unit waveforms;
correcting the time waveform based on the reference waveform; and
acquiring biometric information based on the corrected time waveform.
